(19) United States
(12) Patent Application Publication
(10) Pub. No.: US 2002/0022742 A1
(43) Pub. Date: Feb. 21, 2002

(54) Salt forms of an HIV protease inhibitor

(76) Inventors: Gregory D. Harris, Wilmington, DE (US); Stephen R. Anderson, Wilmington, DE (US); Sridhar Desikan, Wilmington, DE (US); Paul A. Meenan, Bear, DE (US); Benjamin R. Stone, West Chester, PA (US); Pascal H. Toma, Hockessin, DE (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/908,126

(22) Filed: Jul. 18, 2001

Related U.S. Application Data
(60) Provisional application No. 60/219,794, filed on Jul. 19, 2000.

(51) Int. Cl. C07C 311/38

(52) U.S. Cl. 562/45; 562/84; 562/590

(57) ABSTRACT

This invention relates generally to salt forms the compound of formula I:

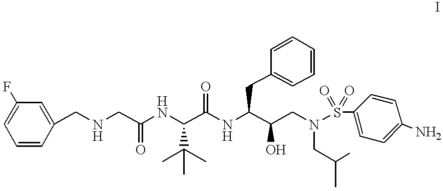

that are useful as HIV protease inhibitors, pharmaceutical compositions comprising the same, and methods of using the same for treating viral infection.

### FIELD OF THE INVENTION

This invention relates generally to salt forms of compound A, described below. The present invention also relates to pharmaceutical compositions comprising the same and methods of using the same.

### BACKGROUND OF THE INVENTION

The present invention relates to salt forms of compound A, shown below.

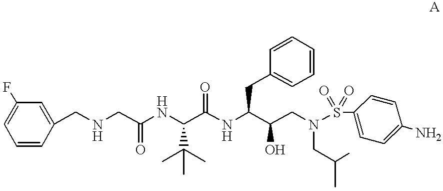

Compound A has been tested and proven to be a potent HIV protease inhibitor. It's bis-hydrochloride salt is disclosed as Example 1 in U.S. Ser. Number 09/482,146, filed Jan. 12, 2000, the contents of which are hereby incorporated by reference.

Compound A has not been known previously to exist in stable crystalline polymorphic forms or in salt forms besides the bis-hydrochloride. For the manufacture, purification, and formulation of drug substances, it is advantageous to discover stable crystalline forms that are either free-base or salt forms of Compound A.

### SUMMARY OF THE INVENTION

Accordingly, one object of the present invention is to provide novel salt forms of Compound A.

It is another object of the present invention to provide pharmaceutical compositions with protease inhibiting activity comprising a pharmaceutically acceptable carrier and a therapeutically effective amount of at least one of the compounds of the present invention or a pharmaceutically acceptable salt form thereof.

It is another object of the present invention to provide a novel method for treating HIV infection which comprises administering to a host in need of such treatment a therapeutically effective amount of at least one of the compounds of the present invention or a pharmaceutically acceptable salt form thereof.

It is another object of the present invention to provide a novel method for treating HIV infection which comprises administering to a host in need thereof a therapeutically effective combination of (a) one of the compounds of the present invention and (b) one or more compounds selected form the group consisting of HIV reverse transcriptase inhibitors and HIV protease inhibitors.

It is another object of the present invention to provide novel salts for use in therapy.

It is another object of the present invention to provide the use of novel salts for the manufacture of a medicament for the treatment of HIV infection.

These and other objects, which will become apparent during the following detailed description, have been achieved by the inventors' discovery that novel salts of the compound of Formula I:

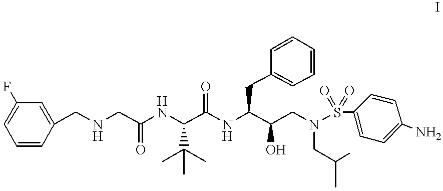

are effective protease inhibitors.

### BRIEF DESCRIPTION OF THE DRAWINGS

The invention is illustrated by reference to the accompanying drawings described below.

FIG. 1 shows a powder x-ray diffractogram of the free base of Compound A isolated from ethyl acetate/heptane.

FIG. 2 shows a differential calorimetry thermogram of the free base of Compound A isolated from ethyl acetate/heptane.

FIG. 3 shows a powder x-ray diffractogram of the mono-fumarate salt of Compound A.

FIG. 4 shows a differential calorimetry thermogram of the mono-fumarate salt of Compound A.

FIG. 5 shows a powder x-ray diffractogram of the mono-(1S)(+)-camphor sulfonate salt of Compound A.

FIG. 6 shows a differential calorimetry thermogram of the mono-(1S)(+)-camphor sulfonate salt of Compound A.

FIG. 7 shows a powder x-ray diffractogram of the mono-methane sulfonate salt of Compound A.

FIG. 8 shows a differential calorimetry thermogram of the mono-methane sulfonate salt of Compound A.

FIG. 9 shows a powder x-ray diffractogram of the mono-phosphate salt of Compound A.

FIG. 10 shows a differential calorimetry thermogram of the mono-phosphate salt of Compound A.

FIG. 11 shows a thermogravimetric thermogram of the mono-phosphate salt of Compound A.

FIG. 12 shows a powder x-ray diffractogram of the bis-p-toluene sulfonate salt of Compound A.

FIG. 13 shows a differential calorimetry thermogram of the bis-p-toluene sulfonate salt of Compound A.

### DETAILED DESCRIPTION OF PREFERRED EMBODIMENTS

Thus, in an embodiment, the present invention provides a novel salt form of the compound of Formula I:

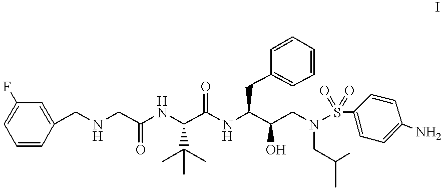

wherein, the salt is selected from mono-fumarate, mono-(1S)(+)-camphor sulfonate, mono-methane sulfonate, mono-phosphate, and bis-toluene-4-sulfonate.

In a preferred embodiment, the present invention provides a novel salt form of the compound of formula I, wherein the salt is the mono-fumarate salt.

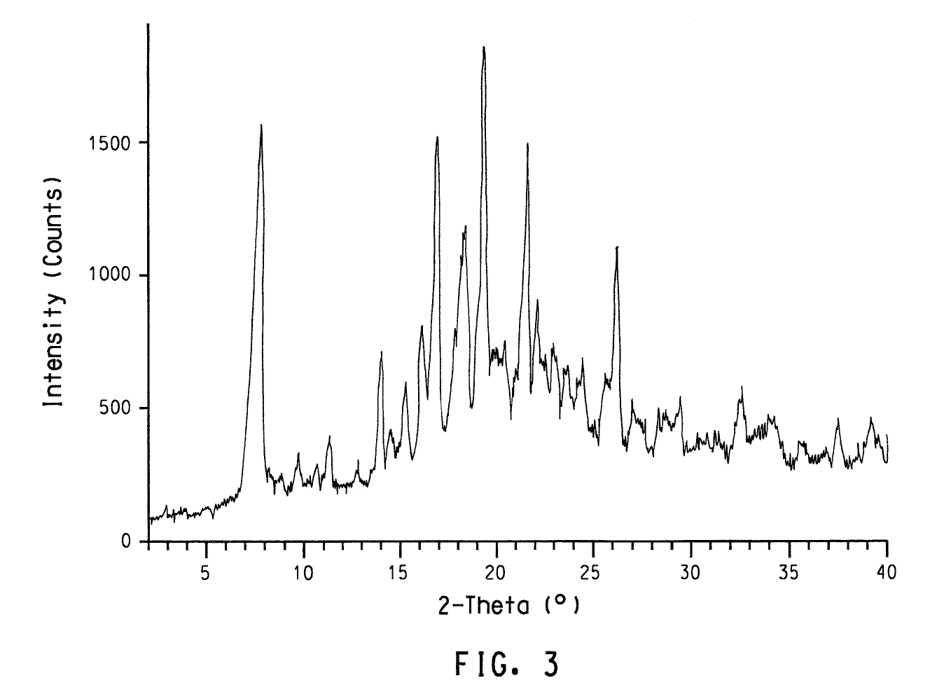

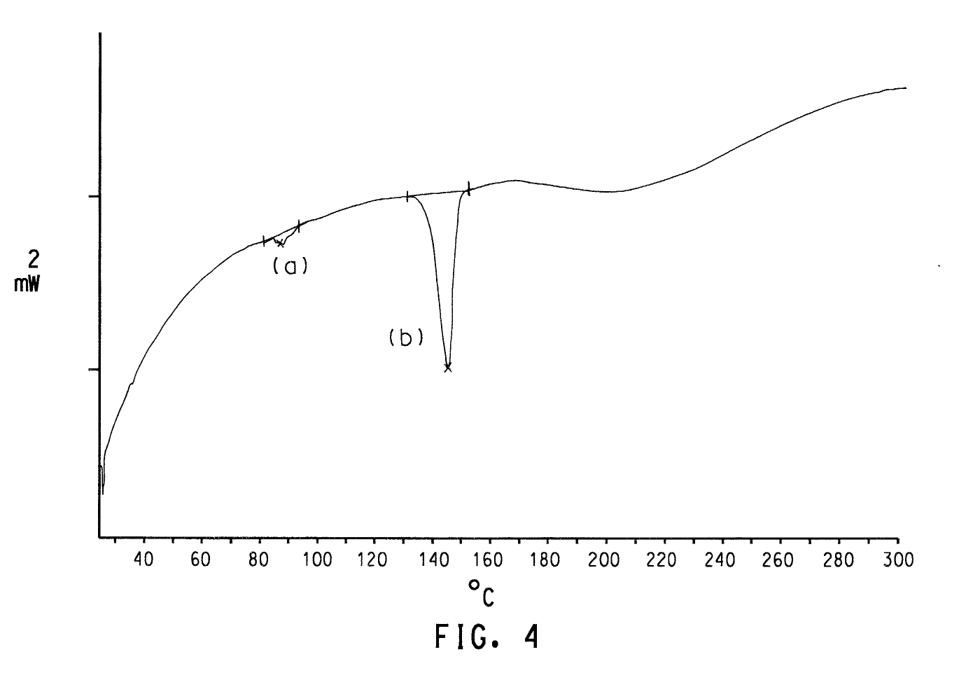

In another preferred embodiment, the mono-fumarate salt is characterized by an x-ray powder diffraction pattern substantially in accordance with that shown in FIG. 3 and a differential scanning calorimetry thermogram substantially in accordance with that shown in FIG. 4.

In another preferred embodiment, the present invention provides a novel salt form of the compound of formula I, wherein the salt is the mono-(1S)(+)-camphor sulfonate salt.

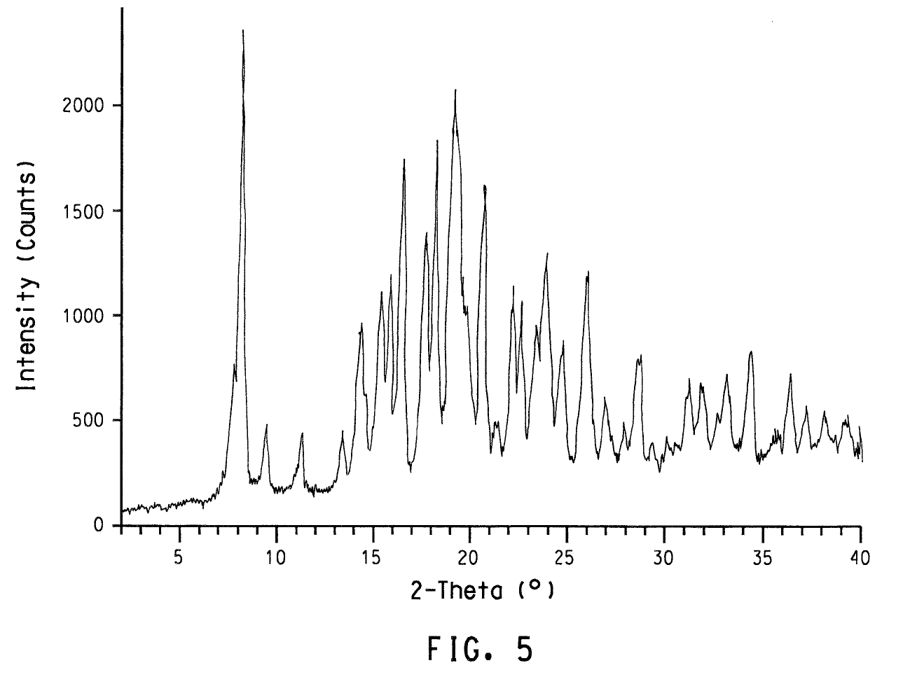

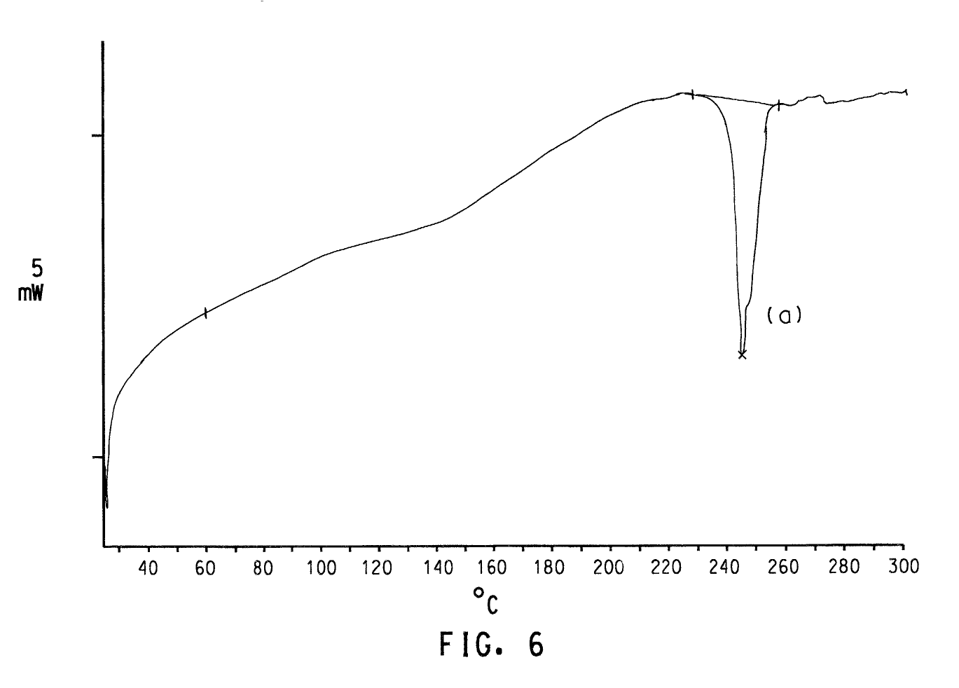

In another preferred embodiment, the mono-(1S)(+)-camphor sulfonate salt is characterized by an x-ray powder diffraction pattern substantially in accordance with that shown in FIG. 5 and a differential scanning calorimetry thermogram substantially in accordance with that shown in FIG. 6.

In another preferred embodiment, the present invention provides a novel salt form of the compound of formula I, wherein the salt is the mono-methane sulfonate salt.

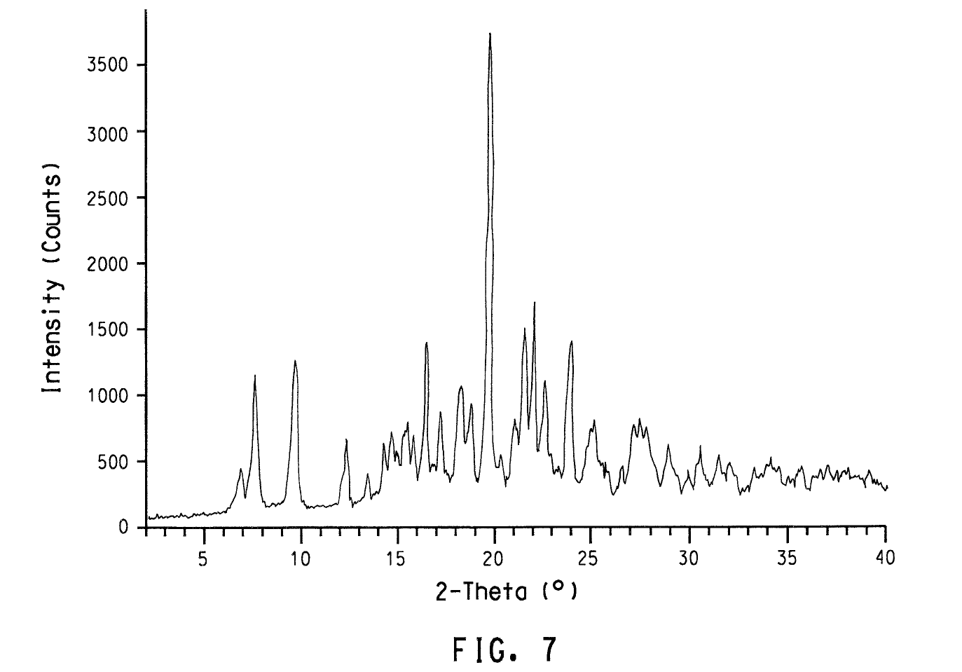

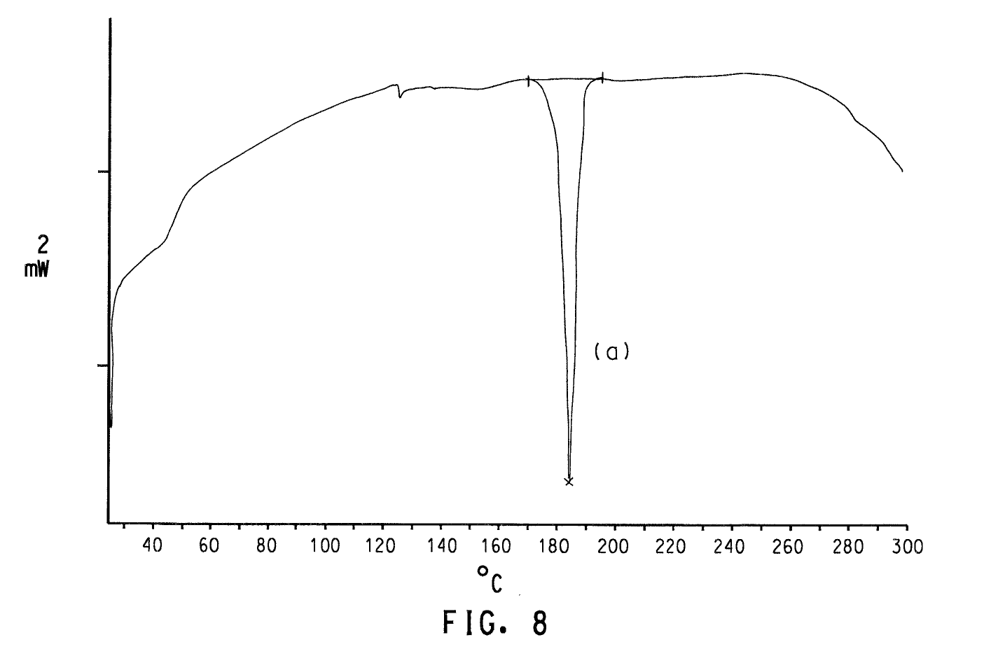

In another preferred embodiment, the mono-methane sulfonate salt is characterized by an x-ray powder diffraction pattern substantially in accordance with that shown in FIG. 7 and a differential scanning calorimetry thermogram substantially in accordance with that shown in FIG. 8.

In another preferred embodiment, the present invention provides a novel salt form of the compound of formula I, wherein the salt is the mono-phosphate salt.

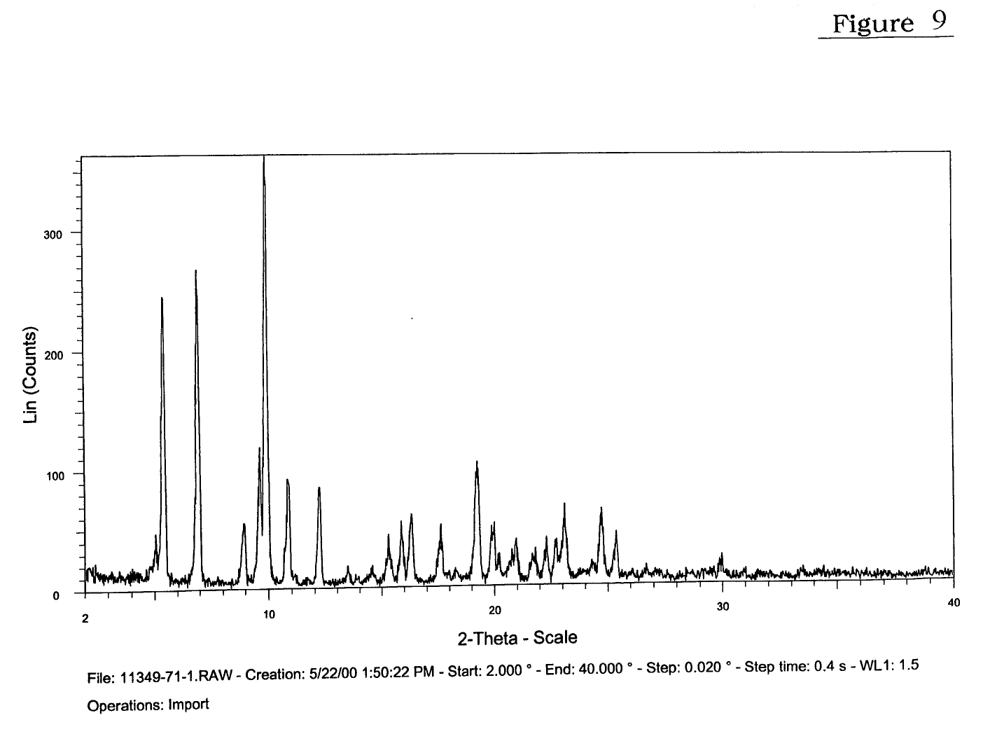

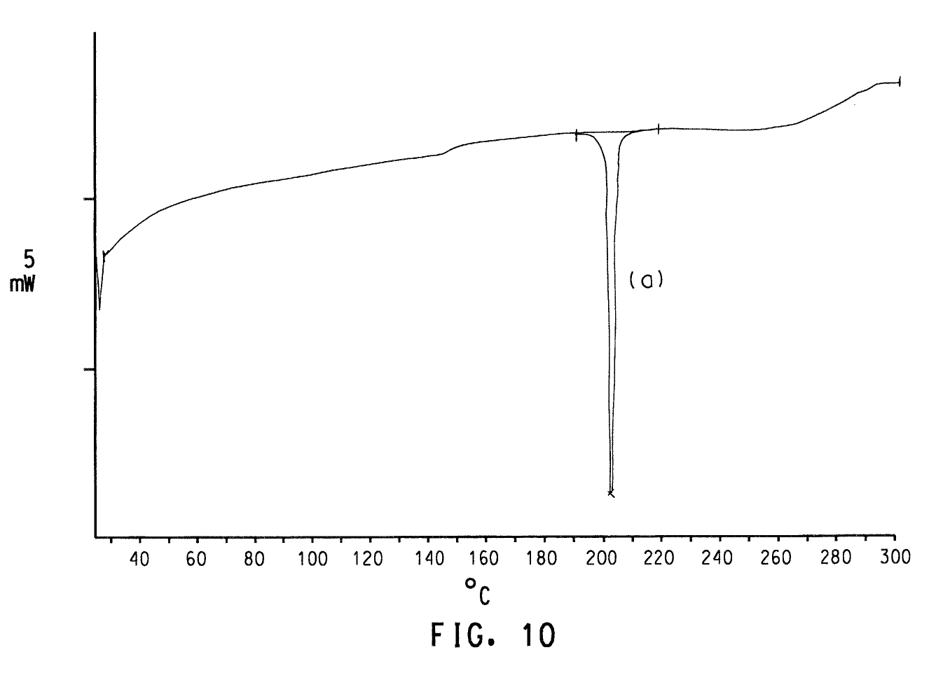

In another preferred embodiment, the mono-phosphate salt is characterized by an x-ray powder diffraction pattern substantially in accordance with that shown in FIG. 9 and a differential scanning calorimetry thermogram substantially in accordance with that shown in FIG. 10.

In another preferred embodiment, the present invention provides a novel salt form of the compound of formula I, wherein the salt is the bis-toluene-4-sulfonate salt.

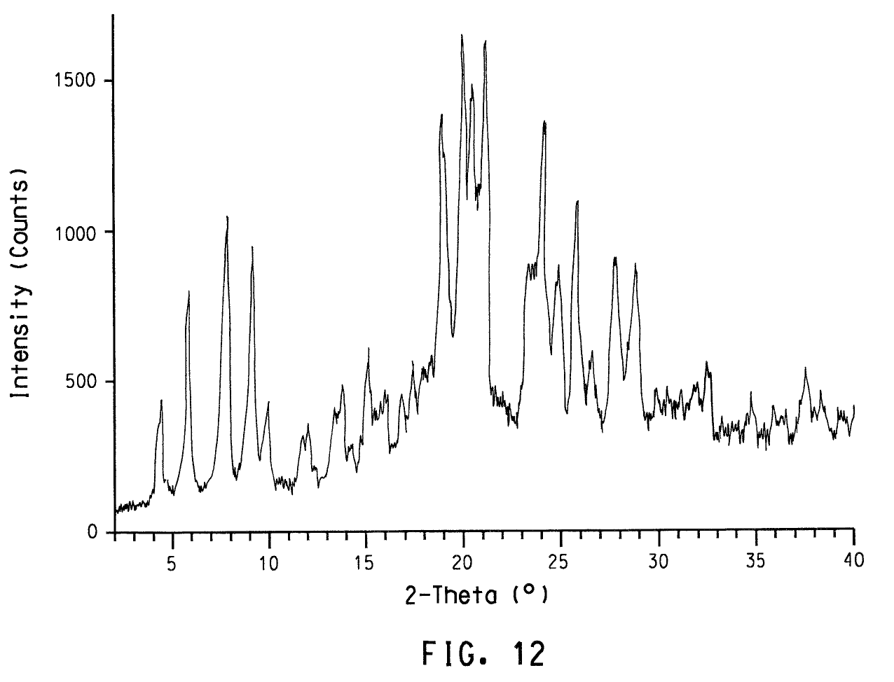

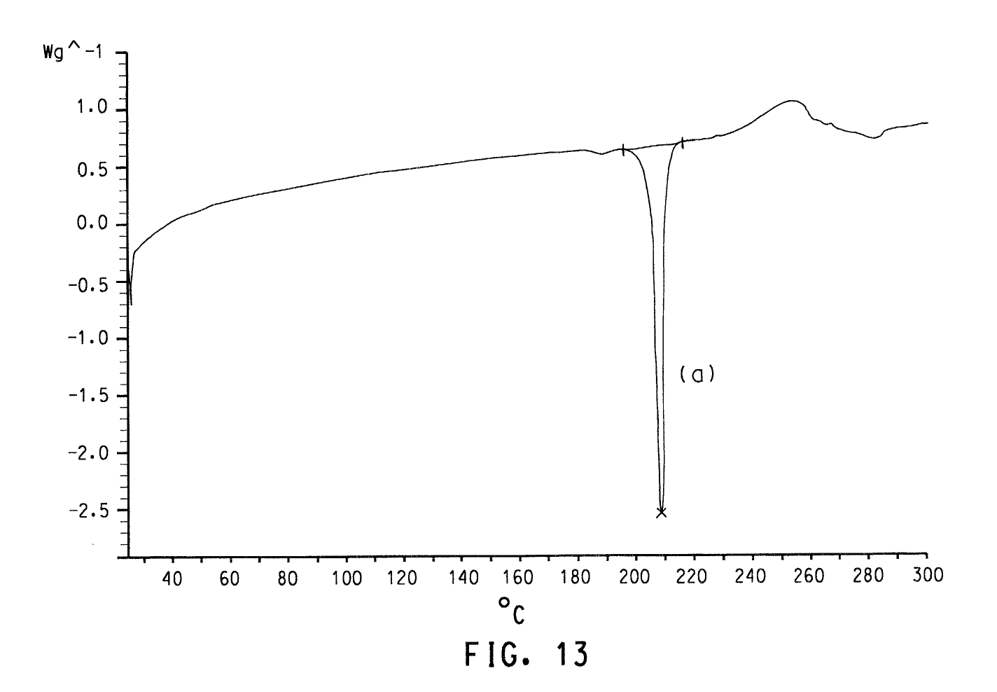

In another preferred embodiment, the bis-toluene-4-sulfonate salt is characterized by an x-ray powder diffraction pattern substantially in accordance with that shown in FIG. 12 and a differential scanning calorimetry thermogram substantially in accordance with that shown in FIG. 13.

In another embodiment, the present invention provides a novel pharmaceutical composition comprising a pharmaceutically acceptable carrier and a therapeutically effective amount of a salt of the present invention.

In another embodiment, the present invention provides a novel method for treating HIV infection that comprises administering to a host in need of such treatment a therapeutically effective amount of a salt of the present invention.

In another embodiment, the present invention provides a novel method of treating HIV infection which comprises administering, in combination, to a host in need thereof a therapeutically effective amount of:

(a) a salt of the present invention; and,

(b) at least one compound selected from the group consisting of HIV reverse transcriptase inhibitors and HIV protease inhibitors.

In another preferred embodiment, the reverse transcriptase inhibitor is selected from the group AZT, ddC, ddI, d4T, 3TC, delavirdine, efavirenz, nevirapine, Ro 18,893, trovirdine, MKC-442, HBY 097, ACT, UC-781, UC-782, RD4-2025, and MEN 10979, and the protease inhibitor is selected from the group saquinavir, ritonavir, indinavir, amprenavir, nelfinavir, palinavir, BMS-232623, GS3333, KNI-413, KNI-272, LG-71350, CGP-61755, PD 173606, PD 177298, PD 178390, PD 178392, U-140690, and ABT-378.

In another preferred embodiment, the reverse transcriptase inhibitor is selected from the group AZT, efavirenz, and 3TC and the protease inhibitor is selected from the group saquinavir, ritonavir, nelfinavir, and indinavir.

In another preferred embodiment, the reverse transcriptase inhibitor is AZT.

In another preferred embodiment, the protease inhibitor is ritonavir.

In another preferred embodiment, component (b) is a HIV reverse transcriptase inhibitor and a HIV protease inhibitor.

In another preferred embodiment, component (b) is two different HIV reverse transcriptase inhibitors.

In another embodiment, the present invention provides a pharmaceutical composition useful for the treatment of HIV infection, which comprises a therapeutically effective amount of:

(a) a salt of the present invention; and,

(b) at least one compound selected from the group consisting of HIV reverse transcriptase inhibitors and HIV protease inhibitors, in one or more sterile containers.

In another embodiment, the present invention provides novel salts for use in therapy.

In another embodiment, the present invention provides the use of novel salts for the manufacture of a medicament for the treatment of HIV.

### Definitions

As used herein, the following terms and expressions have the indicated meanings. It will be appreciated that the compounds of the present invention contain asymmetrically substituted carbon atoms, and may be isolated in optically active or racemic forms. It is well known in the art how to prepare optically active forms, such as by resolution of racemic forms or by synthesis, from optically active starting materials. All chiral, diastereomeric, racemic forms and all geometric isomeric forms of a structure are intended, unless the specific stereochemistry or isomer form is specifically indicated.

The processes of the present invention are contemplated to be practiced on at least a multigram scale, kilogram scale, multikilogram scale, or industrial scale. Multigram scale, as used herein, is preferably the scale wherein at least one starting material is present in 10 grams or more, more preferably at least 50 grams or more, even more preferably at least 100 grams or more. Multikilogram scale, as used herein, is intended to mean the scale wherein more than one kilogram of at least one starting material is used. Industrial scale as used herein is intended to mean a scale which is other than a laboratory scale and which is sufficient to supply product sufficient for either clinical tests or distribution to consumers.

The present invention is intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

The present invention describes compounds in substantially pure form. “Substantially pure” as used herein is intended to mean at least 90, 91, 92, 93, 94, 95, 96, 97, 98, 99, to 100% pure.

For x-ray diffraction, the present invention is intended to encompass compounds yielding diffractograms that are “substantially in accordance” with those presently shown. A diffractogram “substantially in accordance” would be one that comprises 4, 5, 6, 7, 8, 9, 10, 12, 14, 16, 18, 20, 22, 24, 26, 28, 30, 32, 34, 36, 38, 40 or more of the peaks (i.e, 2θ values) within experimental error. Preferably, it would contain ten or more of the peaks. More preferably, it would contain twenty or more of the peaks. Even more preferably, it would contain thirty or more of the peaks. Alternatively, “substantially in accordance” is intended to mean a diffractogram having 20, 25, 30, 35, 40, 45, 50, 55, 60, 65, 70, 75, 80, 85, 90, 95% or more of the same peaks within experimental error. The relative intensities of the peaks may vary, depending upon the sample preparation technique, the sample mounting procedure and the particular instrument employed. Moreover, instrument variation and other factors may affect the 2θ values. Therefore, peak assignments inherently include experimental error and may vary by plus or minus 0.2.

For differential scanning calorimetry (DSC), it is known that the temperatures observed will depend upon the rate of temperature change as well as sample preparation technique and the particular instrument employed. Thus, the values shown in the thermograms may vary by plus or minus 40° C. A thermogram “substantially in accordance” would be one whose peaks vary by plus or minus 4° C. As used herein, “HIV reverse transcriptase inhibitor” is intended to refer to both nucleoside and non-nucleoside inhibitors of HIV reverse transcriptase (RT). Examples of nucleoside RT inhibitors include, but are not limited to, AZT, ddC, ddI, d4T, and 3TC. Examples of non-nucleoside RT inhibitors include, but are not limited to, delavirdine (Pharmacia and Upjohn, U90152S), efavirenz (DuPont), nevirapine (Boehringer Ingelheim), Ro 18,893 (Roche), trovirdine (Lilly), MKC-442 (Triangle), HBY 097 (Hoechst), HBY 1293 (Hoechst), ACT (Korean Research Institute), UC-781 (Rega Institute), UC-782 (Rega Institute), RD4-2025 (Tosoh Co. Ltd.), and MEN 10979 (Menarini Farmaceutici). As used herein, “HIV protease inhibitor” is intended to refer to compounds that inhibit HIV protease. Examples include, but are not limited, saquinavir (Roche, Ro3l-8959), ritonavir (Abbott, ABT-538), indinavir (Merck, MK-639), amprenavir (Vertex/Glaxo Wellcome), nelfinavir (Agouron, AG-1343), palinavir (Boehringer Ingelheim), BMS-232623 (Bristol-Myers Squibb), GS3333 (Gilead Sciences), KNI-413 (Japan Energy), KNI-272 (Japan Energy), LG-71350 (LG Chemical), CGP-61755 (Ciba-Geigy), PD 173606 (Parke Davis), PD 177298 (Parke Davis), PD 178390 (Parke Davis), PD 178392 (Parke Davis), tipranavir (Pharmacia and Upjohn, U-140690), DMP-450 (DuPont) and ABT-378.

“Therapeutically effective amount” is intended to include an amount of a compound of the present invention or an amount of the combination of compounds claimed effective to inhibit HIV infection or treat the symptoms of HIV infection in a host. The combination of compounds is preferably a synergistic combination. Synergy, as described for example by Chou and Talalay, Adv. Enzyme Regul. 22:27-55 (1984), occurs when the effect (in this case, inhibition of HIV replication) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antiviral effect, or some other beneficial effect of the combination compared with the individual components.

Other features of the invention will become apparent in the course of the following descriptions of exemplary embodiments that are given for illustration of the invention and are not intended to be limiting thereof.

### EXAMPLES

Abbreviations used in the Examples are defined as follows: “° C.” for degrees Celsius, “d” for doublet, “dd” for doublet of doublets, “eq” for equivalent or equivalents, “g” for gram or grams, “mg” for milligram or milligrams, “mL” for milliliter or milliliters, “H” for hydrogen or hydrogens, “hr” for hour or hours, “m” for multiplet, “M” for molar, “min” for minute or minutes, “MHz” for megahertz, “MS” for mass spectroscopy, “nmr” or “NMR” for nuclear magnetic resonance spectroscopy, “t” for triplet, and “TLC” for thin layer chromatography.

### Analytical Methods

### X-Ray Powder Diffraction

A uniformly thin layer of solid is spread on a sample holder, and the XRPD is obtained from 2 to 40 degrees 2θ with step size of 0.02 degrees and step time of 0.4 sec.

### Differential Scanning Calorimetry (DSC)

Accurate amount of solids (5 to 15 mg) is weighed in a standard aluminum pan. The sample is covered with a pin-holed aluminum cover. Sample is heated at the rate of 10° C./min. Melting point is reported as the onset of the endotherm in the DSC thermogram.

### Thermogravimetry (TGA)

Accurate amount of solids (1 to 15 mg) is weighed in a ceramic pan. Sample is heated at the rate of 10° C./min with a nitrogen flow of 45 mL/min. Weight loss as a function of temperature is recorded.

### Example 1

### Preparation of Compound A

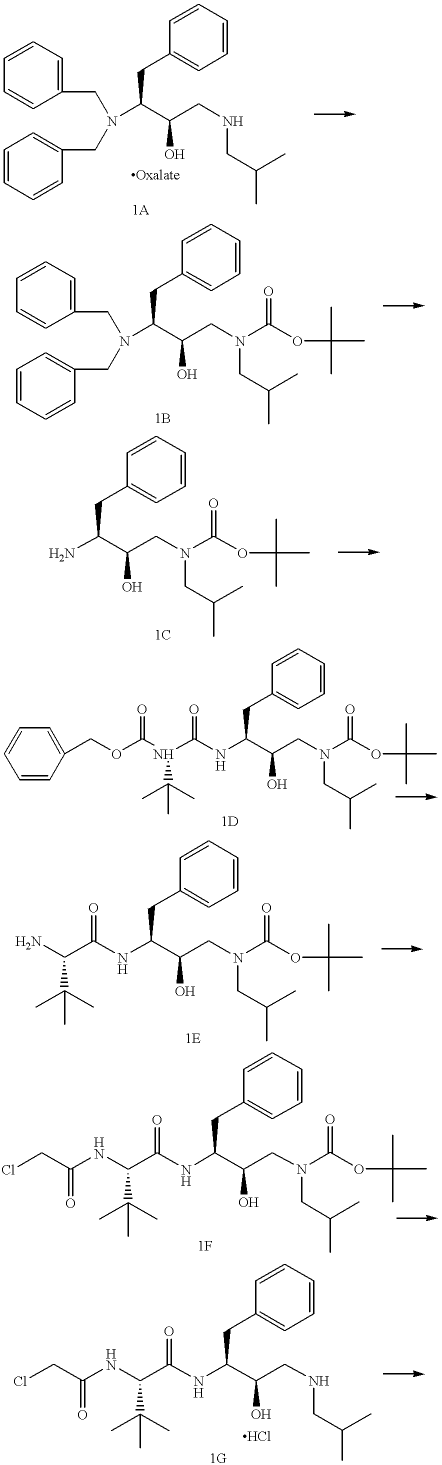

1B To a solution of N-[3(S)-[N,N-bis(phenylmethyl)amino]-2(R)-hydroxy-4-phenylbutyl]-N-isobutylamine.oxalic acid salt 1A (127.6 g, 251 mmol) in toluene (1 L), water (500 mL) and CH₂Cl₂ (400 mL) was added NaOH (50% aqueous, 44.5 g). After stirring 10 min the reaction mixture was extracted with toluene. The combined organic layers were washed with brine, dried (MgSO₄) and the solvent was removed under reduced pressure. The residue was taken up in THF (1 L), cooled to 0° C., and was treated with triethylamine (28.15 g, 278 mmol) and di-tert-butyl dicarbonate (55.23 g, 253 mmol). The solution was warmed to room temperature and was stirred overnight. The solvent was removed under reduced pressure and the residue was taken up in EtOAc (1 L), washed with water, 5% citric acid, water, saturated NaHCO₃, brine, and was dried (MgSO₄). The solvent was removed under reduced pressure to give the carbamate 1B that was used directly without further purification. CIMS (NH3) m/z: 517 (M+H⁺, 100%) 1C To a solution of crude 1B (251 mmol possible) in methanol (500 mL) was added palladium hydroxide on carbon (20%, 10 g). The suspension was placed in a parr bottle and was charged with hydrogen (55 psi). After shaking overnight the reaction mixture was filtered through Celite® and the solvent was removed under reduced pressure. The resulting solid was recrystallized (EtOAc/hexane) to give the amine 1C as a white solid (56.6 g, 67% (2 steps)): CIMS (NH₃) m/z: 337 (M+H⁺, 100%)

1D To a solution of N-carbobenzyloxy-L-tert-leucine (47.5 g, 179 mmol) in DMF (250 mL) at 0° C. was added N-hydroxybenzotriazole (38.6 g, 285 mmol) and EDC (35.7 g, 186 mmol). After stirring 1.5 hours the solution was added to a suspension of 1C (56.6 g, 167 mmol) and 4-methylmorpholine (52.9 g, 521 mmol) in DMF (200 mL). The reaction mixture was allowed to warm to room temperature. After stirring overnight N,N-dimethylethylenediamine (4 mL) was added, the solution was stirred 1.5 hours and the solvent was removed under reduced pressure. The residue was taken up in EtOAc (1 L), washed with water, 5% citric acid, water, saturated NaHCO₃, brine, and was dried (MgSO₄). The solvent was removed under reduced pressure to give 1D (97.5 g, 100%) that was used without further purification. CIMS (NH₃) m/z: 584 (M+H⁺, 100%)

1E To a solution of 1D (97.5 g, 167 mmol) in methanol (300 mL) was added palladium hydroxide on carbon (20%, 10 g). The suspension was placed in a Parr bottle and was charged with hydrogen (55 psi). After shaking overnight the reaction mixture was filtered through Celite® and the solvent was removed under reduced pressure. The resulting solid was recrystallized (EtOAc/hexane) to give the amine 1E as a white solid (72.8 g, 97%): CIMS (NH₃) m/z: 450 (M+H⁺, 100%)

1F To a solution of amine 1E (43.8 g, 97.6 mmol) in EtOAc (400 mL) and water (270 mL) was added KHCO₃ (27.7 g, 276 mmol) and cloroacetyl chloride (12.4 g, 111 mmol). After stirring 3 hours, EtOAc (1 L) was added and the solution was washed with water, 5% citric acid, water, saturated NaHCO₃, brine, and was dried (MgSO₄). The solvent was removed under reduced pressure to give 1F as a white solid (51.0 g, 99%): CIMS (NH₃) m/z: 526 (M+H⁺, 100%)

1G To a solution of 1F (33.8 g, 64.2 mmol) in EtOAc (600 mL) was added 4N HCl in dioxane (80 mL, 320 mmol) and the reaction mixture was stirred 6 hours. The solvent was removed under reduced pressure and the resulting solid was triturated with cold ether to give the hydrochloride salt 1G (28.75 g, 97%): CIMS (NH₃) m/z: 426 (M+H⁺, 100%)

1H To a solution of the salt 1G (32.0 g, 69.2 mmol) in THF (350 mL) and water (450 mL) was added K₂CO₃ (56.7 g, 411 mmol) and 4-nitrobenzenesulfonyl chloride (16.9 g, 76.0 mmol). After stirring 4 hours, water was added and the suspension was extracted with EtOAc. The combined organic layers were washed with brine, 5% citric acid, water, saturated NaHCO₃, brine, and was dried (MgSO₄). The solvent was removed under reduced pressure and the resulting solid was recrystallized (EtOAc/hexane) to give the sulfonamide 1H as a white solid (35.8 g, 85%). CIMS (NH₃) m/z: 611 (M+H⁺, 100%).

1I To a solution of the chloride 1H (16.0 g, 26.1 mmol) in THF (200 mL) was added 3-fluorobenzylamine (20.0 g, 160 mmol) and the reaction mixture was refluxed overnight. The solvent was removed under reduced pressure and the residue was taken up in EtOAc and was washed with water, brine, and dried (MgSO₄). The solvent was removed under reduced pressure and the residue was chromatographed (silica gel, 4% methanol/CH₂Cl₂) to give the amine 1I as a white solid (16.3 g, 89%). CIMS (NH₃) m/z: 700 (M+H⁺, 100%).

1 To a solution of 1I (14.6 g, 20.8 mmol) in methanol (500 mL) was added palladium hydroxide on carbon (20%, 1.5 g) and the reaction mixture was charged with hydrogen. After stirring 3 hours, the mixture was filtered through Celite® and the solvent was removed under reduced pressure. The residue was chromatographed (silica gel, 5% methanol/CH₂Cl₂) to give the amine as a white solid (13.2 g, 95%).

### Example 2

### Preparation of the Free Base Isolated from MTBE/EtOAc

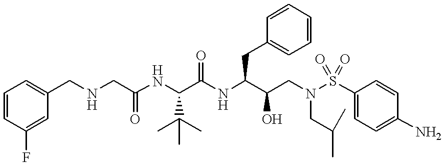

To a slurry of the (1S)(+)-camphor sulfonate salt (34.8 g, 38.57 mmol) in ethyl acetate (250 mL) was added a solution of potassium carbonate (10.65 g, 2 eq) in water (100 mL). The mixture was stirred until there were two clear phases. The phases were separated, the aqueous phase discarded, and the organic phase concentrated to an oil phase. The oil was dissolved in ethyl acetate (30 mL) and methyl tert-butyl ether (300 mL) was added. The resulting slurry was stirred at room temperature for ˜2.5 hours, filtered, and dried to a constant weight in vacuo to give 24.42 g (95%).

### Example 3

### Preparation of the Free Base Isolated from EtOAc/Heptane

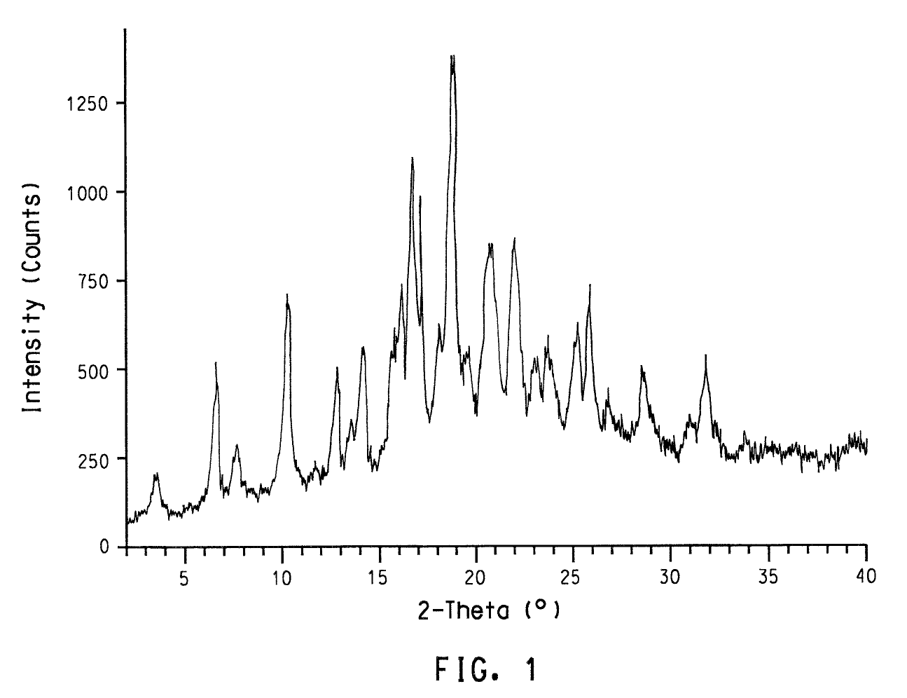

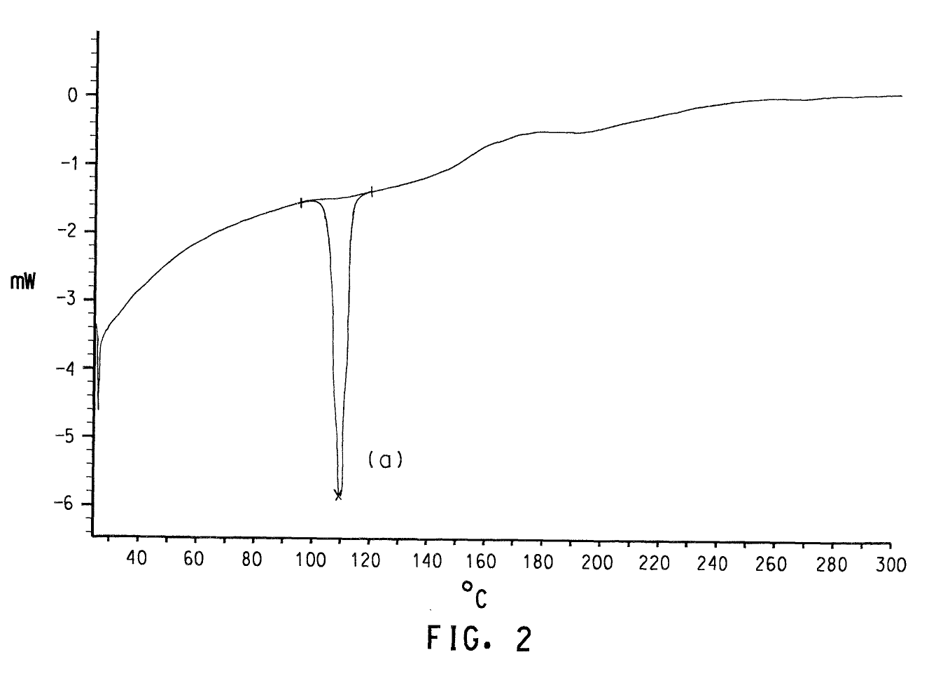

The free base (21.7 g, 32.39 mmol) was dissolved in ethyl acetate (108 mL) and heated to reflux. Heptane (108 mL) was added, the solution cooled to 68° C., seeded (˜0.2 g), cooled to 20° C., and then stirred overnight. The resulting slurry was filtered and dried to a constant weight in vacuo to give 20.2 g (93%). Melting point: 107±5° C. The x-ray diffractogram and differential calorimetry thermogram are shown in FIGS. 1 and 2. Elemental calc: C, 62.76; H, 7.22; F, 2.84; N, 10.46; S, 4.79, found: C, 62.57, H, 7.24, F, 2.90, N, 10.30, S, 4.74. The diffractogram exhibits 2θ values of 3.6±0.2, 6.5±0.2, 7.6±0.2, 10.1 ±0.2, 11.6±0.2, 12.7±0.2, 14.0±0.2, 15.4±0.2, 16.0 ±0.2, 16.5±0.2, 16.9±0.2, 17.9±0.2, 18.7±0.2, 19.4 ±0.2, 20.4±0.2, 20.7±0.2, 21.9±0.2, 22.9±0.2, 23.6 ±0.2, 23.9±0.2, 25.2±0.2, 25.8±0.2, 26.8±0.2, 28.5 ±0.2, 31.7±0.2, 33.7±0.2, and 33.8±0.2.

¹H NMR (400 MHz, CD₃OD). δ0.84 (m, 15 H), 1.94 (m, 1 H), 2.59 (dd, J=2.4, 24.2 Hz, 1 H), 2.83 (m, 1 H), 2.94 (m, 2 H), 3.10-3.18 (m, 3 H), 3.34 (dd, J=l, 15 Hz, 1 H), 3.60 (m, 2 H), 3.79, (m, 1 H), 4.11 (m, 2 H), 6.68 (m, 2 H), 6.68 (d, J=8.4 Hz, 1 H), 6.96 (m, 1 H), 7.02-7.19 (m, 7 H), 7. 30 (m, 1 H), 7.47 (d, J=9.2 Hz, 1 H).

¹³C NMR (100 MHz, CD₃0D). δ20.94, 21.03, 35.86, 37.05, 52.52, 54.29, 54.66, 55.73, 59.71, 62.07, 74.08, 114.88, 115.17, 115.38, 116.28, 116.49, 125.59, 125.62, 126.41, 127.29,129.57, 130.87, 130.98, 131.55, 131.63, 140.33, 144.10, 144.17, 154.72, 163.59, 166.02, 172.40, 173.74

### Example 4

### Preparation of the Mono Fumarate Salt

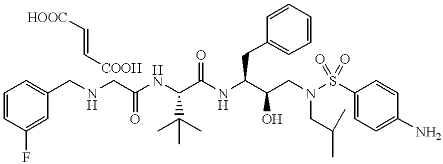

The free base (20.0 g, 29.86 mmol) was slurried in isopropyl alcohol and heated to 50 to 60° C. Fumaric acid (3.46 g, 1 eq) was added. The resulting solution crystallized on cooling to room temperature and was filtered and dried to a constant weight in vacuo at 50° C. to give 18.9 g of the mono-Fumarate salt. The x-ray diffractogram and differential calorimetry thermogram are shown in FIGS. 3 and 4. Melting point: 138±4° C. Elemental calc: C, 59.60; H, 6.67; F, 2.42; N, 8.91; S, 4.08, found: C, 59.24, H, 6.58, F, 2.49, N, 8.71, S, 4.10. The diffractogram exhibits 2θ values of 2.9±0.2, 7.7±0.2, 9.7±0.2, 10.6 ±0.2, 11.3±0.2, 12.8±0.2, 14.0±0.2, 14.5±0.2, 15.3 ±0.2, 16.1±0.2, 16.8±0.2, 18.3±0.2, 19.3±0.2, 19.9 ±0.2, 20.0±0.2, 20.4±0.2, 21.5±0.2, 22.1±0.2, 22.6 ±0.2, 23.0±0.2, 23.7±0.2, 24.2±0.2, 24.5±0.2, 25.7 ±0.2, 26.3±0.2, 27.1±0.2, 27.7±0.2, 28.4±0.2, 28.9 ±0.2, 29.5±0.2, 31.5±0.2, 32.4±0.2, 32.6±0.2, 34.0 ±0.2, 34.3±0.2, 35.5±0.2, 35.9±0.2, 37.5±0.2, 39.1 ±0.2, and 39.5±0.2.

¹H NMR (400 MHz, CD₃OD). δ0.78-0.87 (m, 15 H), 1.93 (m, 1 H), 2.63 (dd, J=3.2, 14.4 Hz, 1 H), 2.81 (m, 1 H), 2.92 (m, 2 H), 3.12 (dd, J=4.2, 14.3, 1 H), 3.38 (m, 1 H), 3.53 (AB, J=16.2, 31.4, 2 H), 3.81 (m, 1 H), 3.97 (m, 2 H), 4.13 (m, 2 H), 6.68 (m, 3 H), 7.04 (m, 1 H), 7.08-7.22 (m, 7 H), 7.39 (m, 1 H), 7.47 (d, J=9.1 Hz, 2 H), 7.96 (d, J=8.5 Hz, 1 H).

¹³C NMR (100 MHz, CD₃OD). δ20.92, 21.01, 27.52, 28.65, 35.64, 36.82, 52.73, 54.53, 55.59, 59.66, 62.86, 74.24, 114.84, 116.90, 117.12, 117.51, 117.73, 126.37, 126.77, 127.50, 129.58, 130.89, 130.96, 132.24, 132.32, 136.24, 140.42, 154.76, 170.52, 172.24.

### Example 5

### Preparation of the Mono (1S)(+)-camphor Sulfonate Salt

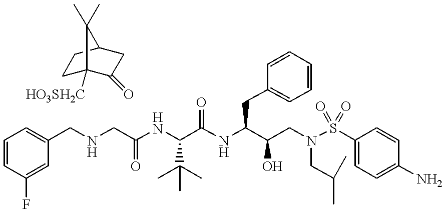

The free base (11.61 g, 17.33 mmol) was dissolved in ethyl acetate (110 mL) at room temperature. (1S)(+)-camphor sulphonic acid (4.02 g, 1 eq) was added. The salt immediately precipitated. Methanol (˜60 mL) was added and the slurry was heated to reflux, cooled to room temperature, filtered and dried in vacuo to a constant weight to give 11.5 g (77%). The x-ray diffractogram and differential calorimetry thermogram are shown in FIGS. 5 and 6. Melting point: 241±4° C. Elemental calc: C, 59.91; H, 7.15; F, 2.11; N, 7.76; S, 7.11, found: C, 59.75, H, 7.16, F, 2.15, N, 7.62, S, 7.09. The diffractogram exhibits 2θ values of 7.6±0.2, 8.1±0.2, 9.4±0.2, 11.2±0.2, 13.3 ±0.2, 14.2±0.2, 15.2±0.2, 15.7±0.2, 16.4±0.2, 17.6 ±0.2, 18.2±0.2, 19.1±0.2, 19.8±0.2, 20.6±0.2, 21.3 ±0.2, 22.2±0.2, 22.6±0.2, 23.3±0.2, 23.8±0.2, 24.7 ±0.2, 26.0±0.2, 27.0±0.2, 27.9±0.2, 28.7±0.2, 29.4 ±0.2, 30.1±0.2, 31.3±0.2, 31.9±0.2, 32.7±0.2, 33.1 ±0.2, 34.3±0.2, 35.5±0.2, 35.8±0.2, 36.4±0.2, 37.2 ±0.2, 38.2±0.2, 39.1±0.2, and 39.3±0.2.

¹H NMR (400 MHz, DMSO d₆). δ073-0.82 (m, 19 H), 1.04 (s, 3 H), 1.25 (m, 2 H), 1.76-1.93 (m, 4 H), 2.24 (m, 1 H), 2.35 (d, J=5.0 Hz, 1 H), 2.63-2.77 (m, 3 H), 2.83-2.97 (m, 3 H), 3.25-3.4 (m, 4 H), 3.60-3.75 (m, 2 H), 4.01 (m, 1 H), 4.17-4.25 (m, 3 H), 4.88 (d, J=9 Hz, 1 H), 4.98 (s, 1 H), 6.59 (d, J=8 Hz, 2 H), 6.99 (m, 1 H), 7.10-7.18 (m, 4 H), 7.28-7.40 (m, 5 H), 7.49-7.54 (m, 1 H), 8.01 (d, J=9 Hz, 1 H), 8.34 (d, J=9, 1 H), 9.26 (s, 1 H).

¹³C NMR (100 MHz, DMSO d₆). δ19.91, 20.42, 20.46, 20.52, 24.49, 26.77, 26.87, 26.92, 34.74, 35.19, 42.49, 42.60, 47.03, 47.39, 49.36, 52.80, 53.49, 57.47, 58.60, 60.13, 71.60, 113.03, 116.30, 116.50, 117.38, 117.60, 123.94, 125.92, 126.88, 128.07, 129.34, 129.64, 131.14, 131.22, 134.28, 134.36, 139.61, 153.11, 161.09, 163.53, 164.53, 169.12.

### Example 6

### Preparation of the Mono-Methane Sulfonate Salt

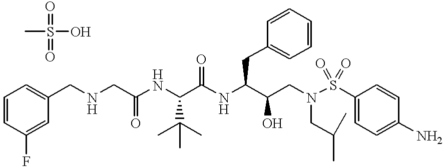

The free base (13.1 g) was dissolved in ethyl propionate (130 mL) and methane sulfonic acid (1.88 g) added. The resulting oily suspension was stirred at 20 to 25° C. for 5 days during which time a white crystalline solid formed. This was filtered and washed with ethyl propionate and dried to constant weight in vacuo at 50° C. Yield 14.9 g. The x-ray diffractogram and differential calorimetry thermogram are shown in FIGS. 7 and 8. Melting point: 181±4° C. Elemental calc: C, 56.45; H, 6.84; N, 9.14; F, 2.48; S, 8.37, found: C, 55.78, H, 6.88, F, 2.52, N, 8.92, S, 8.39. The diffractogram exhibits 2θ values of 6.8±0.2, 7.6±0.2, 9.6±0.2, 12.2±0.2, 13.4±0.2, 14.3±0.2, 14.6±0.2, 15.4±0.2, 15.8±0.2, 16.4±0.2, 17.2±0.2, 18.3±0.2, 18.8±0.2, 19.7±0.2, 20.3±0.2, 21.1±0.2, 21.5±0.2, 22.0±0.2, 22.6±0.2, 23.9±0.2, 25.2±0.2, 25.8±0.2, 26.6±0.2, 27.5±0.2, 27.8±0.2, 28.9±0.2, 29.3±0.2, 30.0±0.2, 30.6±0.2, 31.5±0.2, 32.0±0.2, 32.3±0.2, 33.3±0.2, 34.1±0.2, 34.5±0.2, 35.6±0.2, 36.2±0.2, 36.6±0.2, 36.9±0.2, 37.8±0.2, 38.0±0.2, and 39.0±0.2.

¹H NMR (400 MHz, CD₃OD). δ0.80-0.86 (m, 15 H), 1.93 (m, 1 H), 2.65 (m, 1 H), 2.70 (s, 3 H), 2.77-2.95 (m, 3 H), 3.13 (m, 1 H), 3.41 (dd, J=11.2, 15.2 Hz, 1 H), 3.72-3.87 (m, 3 H), 4.17 (m, 3 H), 6.67 (d, J=8.7 Hz, 2 H), 7.05 (m, 1 H), 7.15-7.26 (m, 7 H), 7.42-7.49 (m, 3H).

¹³C NMR (100 MHz, CD₃OD). δ20.93, 21.03, 27.57, 28.58, 35.42, 36.76, 40.00, 51.83, 54.39, 55.49, 59.56, 63.57, 74.41, 114.90, 118.00, 118.22, 118.34, 118.56, 126.43, 127.53, 127.57, 127.60, 129.63, 130.93, 130.96, 132.69, 132.68, 132.77, 134.87, 134.94, 140.48, 154.73, 163.48, 165.92, 166.24, 172.22.

### Example 7

### Preparation of the Mono Phosphate Salt

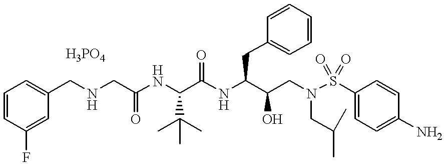

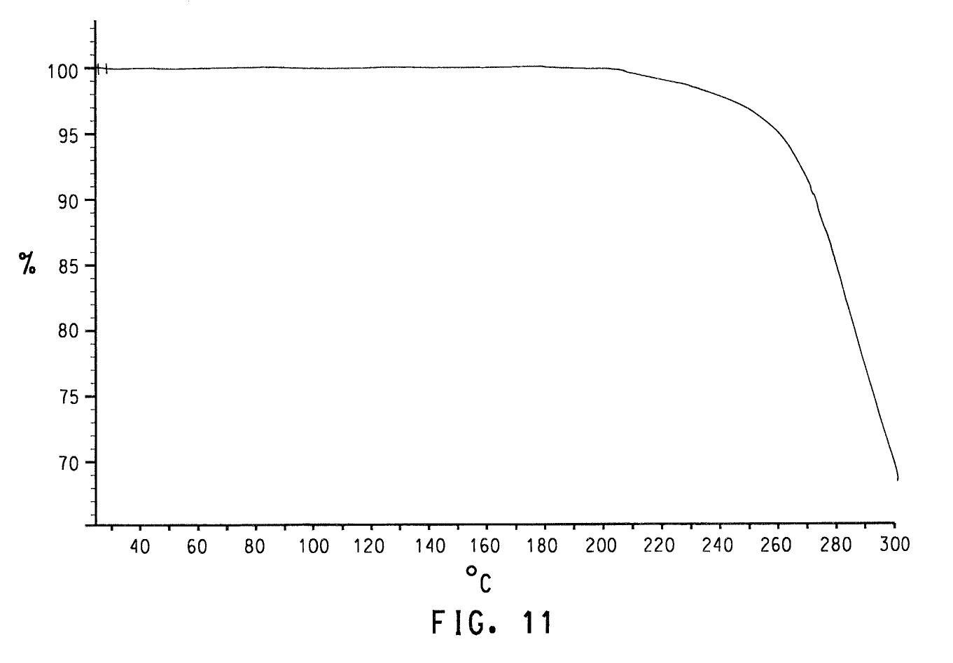

The free base (4.0 g) was charged into a 100 mL jacketed round bottom flask. Absolute ethanol (20 mL) was added and equilibrated at 50° C. One equivalent of anhydrous phosphoric acid dissolved in absolute ethanol (20 mL) was added dropwise at 1 mL per minute. The phosphate salt (100 mg) was added as seed after cooling to 35° C. at 1° C./minute. The slurry was equilibrated for three hours at 10° C. after cooling at 1° C./minute. The slurry was filtered thorough a Buchner funnel and dried at 50° C. to 55° C. for 12 to 18 hours. The x-ray diffractogram, differential calorimetry thermogram, and thermogravimetric thermogram are shown in FIGS. 9, 10, and 11. Melting point: 201±4° C. Elemental calc: C, 54.75; H, 6.69; F, 2.47; N, 9.12; P, 4.03; S, 4.18, found: C, 54.53, H, 6.76, F, 2.57, N, 9.02, P, 4.16, S, 4.32. The diffractogram exhibits 2θ values of 5.4±0.2, 6.9±0.2, 8.9±0.2, 9.6±0.2, 9.9±0.2, 10.8 ±0.2, 12.2±0.2, 13.4±0.2, 14.5±0.2, 15.3±0.2, 15.8 ±0.2, 17.6±0.2, 18.3±0.2, 19.2±0.2, 19.9±0.2, 20.9 ±0.2, 21.7±0.2, 22.3±0.2, 22.7±0.2, 23.1±0.2, 24.7 ±0.2, 25.3±0.2, and 30.0±0.2.

¹H NMR (400 MHz, CD₃OD). δ0.81-0.87 (m, 15 H), 1.94 (m, 1 H), 2.65 (dd, J=11.1, 14.3 Hz, 1 H), 2.80 (m, 1 H), 2.90 (m, 2 H), 3.12 (m, 1 H), 3.39 (dd, J=4.0 Hz, 14.5 Hz, 1 H), 3.72 (AB, J=15.6, 36.9 Hz, 2 H), 3.82 (m, 1 H), 4.80 (m, 2 H), 4.17 (m, 2 H), 6.68 (d, J=8.6 Hz, 2 H), 7.05 (m, 1 H), 7.24 (m, 7 H), 7.42 (m, 1 H), 7.46 (d, J=8.6 Hz, 2 H).

¹³C NMR (100 MHz, CD₃OD). δ20.94, 21.02, 27.56, 28.64, 35.54, 36.68, 52.16, 54.50, 55.52, 59.64, 63.25, 74.29, 114.86, 117.41, 117.62, 118.02, 118.24, 126.36, 127.29, 127.32, 127.54, 129.61, 130.90, 130.97, 132.42, 132.50, 136.66, 140.44, 154.76, 163.78, 165.93, 167.64, 172.21. Alternatively, the mono-phosphate salt is made as follows:

The free base (60 g, 89.6 mmol) was dissolved in isopropanol (400 mL) and warmed to 60° C. Phosphoric acid (85% in water, 1 equivalent, 10.33 g) was added to form a homogeneous solution. Seeds of the phosphate salt were then added (100 mg) and the solution allowed to cool to 20° C. A gelatinous mixture formed which on heating for 30 minutes at reflux transformed to a white crystalline suspension. The mixture was cooled to 20° C., filtered, and dried in vacuo at 50° C. to constant weight (65.8 g, 95%).

### Example 8

### Preparation of the Bis-p-Toluene Sulfonate Salt

The free base (2.00 g, 2.98 mmol) was dissolved in 2-propanol (40 mL). Para-toluene sulfonic acid (1.13 g, 2 eq) was added and the solution stirred overnight at room temperature. The resulting slurry was filtered and dried to a constant weight in vacuo to give 2.35 g. The x-ray diffractogram and differential calorimetry thermogram are shown in FIGS. 12 and 13. Melting point: 206±4° C. Elemental calc: C, 58.03; H, 6.36; F, 1.87; N, 6.90; S, 9.48, found: C, 57.97, H, 6.28, F, 1.93, N, 6.78, S, 9.58. The diffractogram exhibits 2θ values of 4.4±0.2, 5.8±0.2, 7.7±0.2, 9.1±0.2, 9.9±0.2, 11.6±0.2, 12.0±0.2, 13.3±0.2, 13.7±0.2, 15.1±0.2, 16.0±0.2, 16.9±0.2, 17.4±0.2, 18.9±0.2, 20.0±0.2, 20.4±0.2, 21.1±0.2, 23.4±0.2, 24.1±0.2, 24.9±0.2, 25.9±0.2, 26.6±0.2, 27.9±0.2, 28.8±0.2, 30.0±0.2, 30.5±0.2, 31.8±0.2, 32.5±0.2, 33.3±0.2, 34.7±0.2, 35.9±0.2, 36.4±0.2, 37.4±0.2, 38.3±0.2, and 39.2±0.2.

¹H NMR (400 MHz, CD₃OD). δ0.79-0.86 (m, 15 H), 1.96 (m, 1 H), 2.36 (s, 6 H), 2.65 (dd, J=3.0, 14.0 Hz), 2.91 (m, 1 H), 2.94-3.12 (m, 3 H), 3.50 (dd, J=2.8,11.7 Hz, 1 H), 3.78-3.90 (m, 3 H), 4.16 (m, 4 H), 7.04 (m, 1 H), 7.71-7.25 (m, 11 H), 7.40-7.49 (m, 3 H), 7.70 (m, 4 H), 7.93 (d, J=8.6 Hz, 2 H).

¹³C NMR (100 MHz, CD₃OD). δ20.78, 20.82, 21.74, 27.59, 28.28, 35.32, 36.79, 51.81, 53.53, 55.61, 58.62, 63.75, 73.85, 117.98, 118.19, 118.33, 118.56, 124.39, 127.39, 127.57, 129.65, 130.31, 130.89, 130.95, 132.66, 132.74, 134.84, 134.92, 138.56, 140.37, 140.65, 142.24, 143.82, 163.44, 165.89, 166.34, 172.39.

### Utility

The compounds of formula I possess HIV protease inhibitory activity and are therefore useful as antiviral agents for the treatment of HIV infection and associated diseases. The compounds of formula I possess HIV protease inhibitory activity and are effective as inhibitors of HIV growth. The ability of the compounds of the present invention to inhibit viral growth or infectivity is demonstrated in standard assay of viral growth or infectivity, for example, using the assay described below.

As used herein “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar and “nm” denotes nanometer. “Sigma” stands for the Sigma-Aldrich Corp. of St. Louis, Mo.

### HIV RNA Assay

### DNA Plasmids and in vitro RNA Transcripts

Plasmid pDAB 72 containing both gag and pol sequences of BH10 (bp 113-1816) cloned into PTZ 19R was prepared according to Erickson-Viitanen et al. AIDS Research and Human Retroviruses 1989, 5, 577. The plasmid was linearized with Bam HI prior to the generation of in vitro RNA transcripts using the Riboprobe Gemini system II kit (Promega) with T7 RNA polymerase. Synthesized RNA was purified by treatment with RNase free DNAse (Promega), phenol-chloroform extraction, and ethanol precipitation. RNA transcripts were dissolved in water, and stored at −70° C. The concentration of RNA was determined from the A260.

### Probes

Biotinylated capture probes were purified by HPLC after synthesis on an Applied Biosystems (Foster City, Calif.) DNA synthesizer by addition of biotin to the 5′ terminal end of the oligonucleotide, using the biotin-phosphoramidite reagent of Cocuzza, Tet. Lett. 1989, 30, 6287. The gag biotinylated capture probe (5-biotin-CTAGCTCCCTGCTTGCCCATACTA 3′) was complementary to nucleotides 889-912 of HXB2 and the pol biotinylated capture probe (5′-biotin -CCCTATCATTTTTGGTTTCCAT 3′) was complementary to nucleotides 2374-2395 of HXB2. Alkaline phosphatase conjugated oligonucleotides used as reporter probes were prepared by Syngene (San Diego, Calif.). The pol reporter probe (5′ CTGTCTTACTTTGATAAAACCTC 3′) was complementary to nucleotides 2403-2425 of HXB2. The gag reporter probe (5′ CCCAGTATTTGTCTACAGCCTTCT 3′) was complementary to nucleotides 950-973 of HXB2. All nucleotide positions are those of the GenBank Genetic Sequence Data Bank as accessed through the Genetics Computer Group Sequence Analysis Software Package (Devereau Nucleic Acids Research 1984, 12, 387). The reporter probes were prepared as 0.5 μM stocks in 2×SSC (0.3 M NaCl, 0.03 M sodium citrate), 0.05 M Tris pH 8.8, 1 mg/mL BSA. The biotinylated capture probes were prepared as 100 μM stocks in water.

### Streptavidin Coated Plates

Streptavidin coated plates were obtained from Du Pont Biotechnology Systems (Boston, Mass.).

### Cells and Virus Stocks

MT-2 and MT-4 cells were maintained in RPMI 1640 supplemented with 5% fetal calf serum (FCS) for MT-2 cells or 10% FCS for MT-4 cells, 2 mM L-glutamine and 50 μg/mL gentamycin, all from Gibco. HIV-1 RF was propagated in MT-4 cells in the same medium. Virus stocks were prepared approximately 10 days after acute infection of MT-4 cells and stored as aliquots at −70° C. Infectious titers of HIV-1(RF) stocks were 1-3×10⁷ PFU (plaque forming units)/mL as measured by plaque assay on MT-2 cells (see below). Each aliquot of virus stock used for infection was thawed only once.

For evaluation of antiviral efficacy, cells to be infected were subcultured one day prior to infection. On the day of infection, cells were resuspended at 5×10⁵ cells/mL in RPMI 1640, 5% FCS for bulk infections or at 2×10⁶/mL in Dulbecco's modified Eagles medium with 5% FCS for infection in microtiter plates. Virus was added and culture continued for 3 days at 37° C.

### HIV RNA Assay

Cell lysates or purified RNA in 3 M or 5 M GED were mixed with 5 M GED and capture probe to a final guanidinium isothiocyanate concentration of 3 M and a final biotin oligonucleotide concentration of 30 nM. Hybridization was carried out in sealed U bottom 96 well tissue culture plates (Nunc or Costar) for 16-20 hours at 37° C. RNA hybridization reactions were diluted three-fold with deionized water to a final guanidinium isothiocyanate concentration of 1 M and aliquots (150 μL) were transferred to streptavidin coated microtiter plates wells. Binding of capture probe and capture probe-RNA hybrid to the immobilized streptavidin was allowed to proceed for 2 hours at room temperature, after which the plates were washed 6 times with DuPont ELISA plate wash buffer (phosphate buffered saline(PBS), 0.05% Tween 20.) A second hybridization of reporter probe to the immobilized complex of capture probe and hybridized target RNA was carried out in the washed streptavidin coated well by addition of 120 μl of a hybridization cocktail containing 4×SSC, 0.66% Triton X 100, 6.66% deionized formamide, 1 mg/mL BSA and 5 nM reporter probe. After hybridization for one hour at 37° C., the plate was again washed 6 times. Immobilized alkaline phosphatase activity was detected by addition of 100 μL of 0.2 mM 4-methylumbelliferyl phosphate (MUBP, JBL Scientific) in buffer δ(2.5 M diethanolamine pH 8.9 (JBL Scientific), 10 mM MgCl₂, 5 mM zinc acetate dihydrate and 5 mM N-hydroxyethyl-ethylene-diamine-triacetic acid). The plates were incubated at 37° C. Fluorescence at 450 nM was measured using a microplate fluorometer (Dynateck) exciting at 365 nM.

### Microplate Based Compound Evaluation in HIV-1 Infected MT-2 Cells

Compounds to be evaluated were dissolved in DMSO and diluted in culture medium to twice the highest concentration to be tested and a maximum DMSO concentration of 2%. Further three-fold serial dilutions of the compound in culture medium were performed directly in U bottom microtiter plates (Nunc). After compound dilution, MT-2 cells (50 μL) were added to a final concentration of 5×10⁵ per mL (1×10⁵ per well). Cells were incubated with compounds for 30 minutes at 37° C. in a CO₂ incubator. For evaluation of antiviral potency, an appropriate dilution of HIV-1 (RF) virus stock (50 μL) was added to culture wells containing cells and dilutions of the test compounds. The final volume in each well was 200 μL. Eight wells per plate were left uninfected with 50 μL of medium added in place of virus, while eight wells were infected in the absence of any antiviral compound. For evaluation of compound toxicity, parallel plates were cultured without virus infection.

After 3 days of culture at 37° C. in a humidified chamber inside a CO₂ incubator, all but 25 μL of medium/well was removed from the HIV infected plates. Thirty seven μL of 5 M GED containing biotinylated capture probe was added to the settled cells and remaining medium in each well to a final concentration of 3 M GED and 30 nM capture probe. Hybridization of the capture probe to HIV RNA in the cell lysate was carried out in the same microplate well used for virus culture by sealing the plate with a plate sealer (Costar), and incubating for 16-20 hrs in a 37° C. incubator. Distilled water was then added to each well to dilute the hybridization reaction three-fold and 150 μL of this diluted mixture was transferred to a streptavidin coated microtiter plate. HIV RNA was quantitated as described above. A standard curve, prepared by adding known amounts of PDAB 72 in vitro RNA transcript to wells containing lysed uninfected cells, was run on each microtiter plate in order to determine the amount of viral RNA made during the infection.

In order to standardize the virus inoculum used in the evaluation of compounds for antiviral activity, dilutions of virus were selected which resulted in an IC₉₀ value (concentration of compound required to reduce the HIV RNA level by 90%) for dideoxycytidine (ddC) of 0.2 μg/mL. IC₉₀ values of other antiviral compounds, both more and less potent than ddC, were reproducible using several stocks of HIV-1 (RF) when this procedure was followed. This concentration of virus corresponded to ˜3×10⁵ PFU (measured by plaque assay on MT-2 cells) per assay well and typically produced approximately 75% of the maximum viral RNA level achievable at any virus inoculum. For the HIV RNA assay, IC₉₀ values were determined from the percent reduction of net signal (signal from infected cell samples minus signal from uninfected cell samples) in the RNA assay relative to the net signal from infected, untreated cells on the same culture plate (average of eight wells). Valid performance of individual infection and RNA assay tests was judged according to three criteria. It was required that the virus infection should result in an RNA assay signal equal to or greater than the signal generated from 2 ng of pDAB 72 in vitro RNA transcript. The IC₉₀ for ddC, determined in each assay run, should be between 0.1 and 0.3 μg/mL. Finally, the plateau level of viral RNA produced by an effective protease inhibitor should be less than 10% of the level achieved in an uninhibited infection. A compound was considered active if its IC₉₀ was found to be less than 1 μM.

For antiviral potency tests, all manipulations in microtiter plates, following the initial addition of 2X concentrated compound solution to a single row of wells, were performed using a Perkin Elmer/Cetus ProPette.

### Dosage and Formulation

The antiviral compounds of this invention can be administered as treatment for viral infections by any means that produces contact of the active agent with the agent's site of action, i.e., the viral protease, in the body of a mammal. They can be administered by any conventional means available for use in conjunction with pharmaceuticals, either as individual therapeutic agents or in a combination of therapeutic agents. They can be administered alone, but preferably are administered with a pharmaceutical carrier selected on the basis of the chosen route of administration and standard pharmaceutical practice.

The dosage administered will, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to about 1000 milligrams per kilogram of body weight, with the preferred dose being about 0.1 to about 30 mg/kg.

Dosage forms of compositions suitable for administration contain from about 1 mg to about 100 mg of active ingredient per unit. In these pharmaceutical compositions the active ingredient will ordinarily be present in an amount of about 0.5-95% by weight based on the total weight of the composition. The active ingredient can be administered orally in solid dosage forms, such as capsules, tablets and powders, or in liquid dosage forms, such as elixirs, syrups and suspensions. It can also be administered parenterally, in sterile liquid dosage forms.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts, and sodium EDTA. In addition, parenteral solutions can contain preservatives, such as benzalkonium chloride, methyl- or propyl-paraben and chlorobutanol. Suitable pharmaceutical carriers are described in Remington's Pharmaceutical Sciences, supra, a standard reference text in this field.

Useful pharmaceutical dosage-forms for administration of the compounds of this invention can be illustrated as follows:

### Capsules

A large number of unit capsules can be prepared by filling standard two-piece hard gelatin capsules each with 100 mg of powdered active ingredient, 150 mg of lactose, 50 mg of cellulose, and 6 mg magnesium stearic.

### Soft Gelatin Capsules

A mixture of active ingredient in a digestible oil such as soybean oil, cottonseed oil or olive oil can be prepared and injected by means of a positive displacement pump into gelatin to form soft gelatin capsules containing 100 mg of the active ingredient. The capsules should then be washed and dried.

### Tablets

A large number of tablets can be prepared by conventional procedures so that the dosage unit is 100 mg of active ingredient, 0.2 mg of colloidal silicon dioxide, 5 milligrams of magnesium stearate, 275 mg of microcrystalline cellulose, 11 mg of starch and 98.8 mg of lactose. Appropriate coatings may be applied to increase palatability or delay absorption.

### Suspension

An aqueous suspension can be prepared for oral administration so that each 5 mL contain 25 mg of finely divided active ingredient, 200 mg of sodium carboxymethyl cellulose, 5 mg of sodium benzoate, 1.0 g of sorbitol solution, U.S.P., and 0.025 mg of vanillin.

### Injectable

A parenteral composition suitable for administration by injection can be prepared by stirring 1.5% by weight of active ingredient in 10% by volume propylene glycol and water. The solution is sterilized by commonly used techniques.

### Combination of Components (a) and (b)

Each therapeutic agent component of this invention can independently be in any dosage form, such as those described above, and can also be administered in various ways, as described above. In the following description component (b) is to be understood to represent one or more agents as described previously. Thus, if components (a) and (b) are to be treated the same or independently, each agent of component (b) may also be treated the same or independently.

Components (a) and (b) of the present invention may be formulated together, in a single dosage unit (that is, combined together in one capsule, tablet, powder, or liquid, etc.) as a combination product. When component (a) and (b) are not formulated together in a single dosage unit, the component (a) may be administered at the same time as component (b) or in any order; for example component (a) of this invention may be administered first, followed by administration of component (b), or they may be administered in the reverse order. If component (b) contains more that one agent, e.g., one RT inhibitor and one protease inhibitor, these agents may be administered together or in any order. When not administered at the same time, preferably the administration of component (a) and (b) occurs less than about one hour apart. Preferably, the route of administration of component (a) and (b) is oral. The terms oral agent, oral inhibitor, oral compound, or the like, as used herein, denote compounds that may be orally administered. Although it is preferable that component (a) and component (b) both be administered by the same route (that is, for example, both orally) or dosage form, if desired, they may each be administered by different routes (that is, for example, one component of the combination product may be administered orally, and another component may be administered intravenously) or dosage forms.

As is appreciated by a medical practitioner skilled in the art, the dosage of the combination therapy of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

The proper dosage of components (a) and (b) of the present invention will be readily ascertainable by a medical practitioner skilled in the art, based upon the present disclosure. By way of general guidance, typically a daily dosage may be about 100 milligrams to about 1.5 grams of each component. If component (b) represents more than one compound, then typically a daily dosage may be about 100 milligrams to about 1.5 grams of each agent of component (b). By way of general guidance, when the compounds of component (a) and component (b) are administered in combination, the dosage amount of each component may be reduced by about 70-80% relative to the usual dosage of the component when it is administered alone as a single agent for the treatment of HIV infection, in view of the synergistic effect of the combination.

The combination products of this invention may be formulated such that, although the active ingredients are combined in a single dosage unit, the physical contact between the active ingredients is minimized. In order to minimize contact, for example, where the product is orally administered, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose or other appropriate materials as known in the art, in order to further separate the active components. The polymer coating serves to form an additional barrier to interaction with the other component. In each formulation wherein contact is prevented between components (a) and (b) via a coating or some other material, contact may also be prevented between the individual agents of component (b).

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time or concurrently by the same manner, will be readily apparent to those skilled in the art, based on the present disclosure.

Pharmaceutical kits useful for the treatment of HIV infection, which comprise a therapeutically effective amount of a pharmaceutical composition comprising a compound of component (a) and one or more compounds of component (b), in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers of materials may comprise separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Numerous modifications and variations of the present invention are possible in light of the above teachings. It is therefore to be understood that within the scope of the appended claims, the invention may be practiced otherwise than as specifically described herein.

What is claimed:

1. A salt of a compound of formula I: 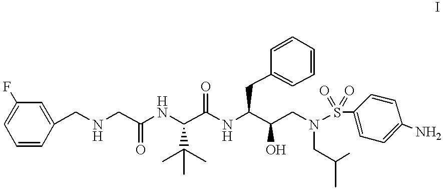
wherein, the salt is selected from mono-fumarate, mono-(1S)(+)-camphor sulfonate, mono-methane sulfonate, mono-phosphate, and bis-toluene-4-sulfonate.

2. A salt according to claim 1, wherein the salt is the mono-fumarate salt.

3. A salt according to claim 2, wherein the mono-fumarate salt is characterized by an x-ray powder diffraction pattern substantially in accordance with that shown in FIG. 3 and a differential scanning calorimetry thermogram substantially in accordance with that shown in FIG. 4.

4. A salt according to claim 1, wherein the salt is the mono-(1S)(+)-camphor sulfonate salt.

5. A salt according to claim 4, wherein the mono-(1S)(+)-camphor sulfonate salt is characterized by an x-ray powder diffraction pattern substantially in accordance with that shown in FIG. 5 and a differential scanning calorimetry thermogram substantially in accordance with that shown in FIG. 6.

6. A salt according to claim 1, wherein the salt is the mono-methane sulfonate salt.

7. A salt according to claim 6, wherein the mono-methane sulfonate salt is characterized by an x-ray powder diffraction pattern substantially in accordance with that shown in FIG. 7 and a differential scanning calorimetry thermogram substantially in accordance with that shown in FIG. 8.

8. A salt according to claim 1, wherein the salt is the mono-phosphate salt.

9. A salt according to claim 8, wherein the mono-phosphate salt is characterized by an x-ray powder diffraction pattern substantially in accordance with that shown in FIG. 9 and a differential scanning calorimetry thermogram substantially in accordance with that shown in FIG. 10.

10. A salt according to claim 1, wherein the salt is the bis-toluene-4-sulfonate salt.

11. A salt according to claim 10, wherein the bis-toluene-4-sulfonate salt is characterized by an x-ray powder diffraction pattern substantially in accordance with that shown in FIG. 12 and a differential scanning calorimetry thermogram substantially in accordance with that shown in FIG. 13.

12. A pharmaceutical composition, comprising: a pharmaceutically acceptable carrier and a therapeutically effective amount of a salt according to claim 1.

13. A method for treating HIV infection, comprising: administering to a host in need of such treatment a therapeutically effective amount of a salt according to claim 1.

14. A method of treating HIV infection which comprises administering, in combination, to a host in need thereof a therapeutically effective amount of:
(a) a salt according to claim 1 and,
(b) at least one compound selected from the group consisting of HIV reverse transcriptase inhibitors and HIV protease inhibitors.

15. A method according to claim 14, wherein the reverse transcriptase inhibitor is selected from the group AZT, ddC, ddI, d4T, 3TC, delavirdine, efavirenz, nevirapine, Ro 18,893, trovirdine, MKC-442, HBY 097, ACT, UC-781, UC-782, RD4-2025, and MEN 10979 and the protease inhibitor is selected from the group saquinavir, ritonavir, indinavir, amprenavir, nelfinavir, palinavir, BMS-232623, GS3333, KNI-413, KNI-272, LG-71350, CGP-61755, PD 173606, PD 177298, PD 178390, PD 178392, U-140690, and ABT-378.

16. A method according to claim 15, wherein the reverse transcriptase inhibitor is selected from the group AZT, efavirenz, and 3TC and the protease inhibitor is selected from the group saquinavir, ritonavir, nelfinavir, and indinavir.

17. A method according to claim 16, wherein compound (b) is ritonavir.
